(19) United States
(12) Patent Application Publication
(10) Pub. No.: US 2024/0369578 A1
(43) Pub. Date: Nov. 7, 2024

(54) PEPTIDE T14 FOR BRAAK STAGING

(71) Applicant: NEURO-BIO LTD, Abingdon, Oxfordshire (GB)

(72) Inventor: Susan GREENFIELD, Abingdon, Oxfordshire (GB)

(21) Appl. No.: 18/690,169

(22) PCT Filed: Sep. 8, 2022
(86) PCT No.: PCT/GB2022/052280

§ 371 Date: Mar. 7, 2024

(30) Foreign Application Priority Data
Sep. 9, 2021 (GB) 2112831.9

(51) Int. Cl. G01N 33/68; C07K 14/47

(52) U.S. Cl. CPC G01N 33/6896; C07K 14/47; G01N 2800/2821; G01N 2800/56

(57) ABSTRACT

The invention relates to biomarkers, and particularly, although not exclusively, to biomarkers for neurodegenerative disorders, such as Alzheimer's disease, Parkinson's disease, Huntington's disease or Motor Neurone disease. The invention especially relates to novel biomarkers for facilitating Braak staging for classifying the degree of pathology in Alzheimer's disease in living patients, and determining the need or otherwise of Positron Emission Topography (PET) scanning for detecting the presence of beta amyloid in the brain. The invention further provides diagnostic and prognostic methods and kits for neurodegenerative disorders, and for Braak staging and determining the need for conducting a PET scan on a subject suspected of suffering from Alzheimer's disease.

The invention relates to biomarkers, and particularly, although not exclusively, to biomarkers for neurodegenerative disorders, such as Alzheimer's disease, Parkinson's disease, Huntington's disease or Motor Neurone disease. The invention especially relates to novel biomarkers for facilitating Braak staging for classifying the degree of pathology in Alzheimer's disease in living patients, and determining the need or otherwise of Positron Emission Topography (PET) scanning for detecting the presence of beta amyloid in the brain. The invention further provides diagnostic and prognostic methods and kits for neurodegenerative disorders, and for Braak staging and determining the need for conducting a PET scan on a subject suspected of suffering from Alzheimer's disease.

Alzheimer's disease (AD) is fast becoming one of the biggest socioeconomic burdens in the world with a growing worldwide incidence rate of almost 10 million new cases of dementia each year. Since both the incidence and prevalence of AD increases with age, the number of patients is growing significantly with an aging population. In 2015, there were over 46 million people living with dementia with an estimated socioeconomic cost of $800 billion annually and it is expected that the number of patients will rise to over 130 million by 2050 costing society over $2 trillion annually. AD was recently announced as the UK's leading cause of death in the over 65's, and in the US it is now the 6th leading cause of death across all ages.

At present, there exists no single test that can be carried out to diagnose Alzheimer's disease, or to prognose cognitive decline. Clinical classification of AD relies on a combination of subjective reporting, medical history evaluation, cognitive function tests, and costly brain imaging scans with no true classification possible until a post-mortem examination of the brain can be conducted.

Braak staging refers to two methods used to classify the degree of pathology in

Alzheimer's disease and Parkinson's disease. These methods are used in research and for the clinical diagnosis of these diseases, and are obtained by performing an autopsy of the brain. Braak staging has six stages based on the location of neurofibrillary tangles with Braak stage 0 corresponding to a healthy or normal (i.e. no disease) individual. Stages I and II relates to early stage AD and is when neurofibrillary tangles are limited to the transentorhinal region of the brain. Stages III and IV define neurofibrillary tangle involvement in the limbic regions, which includes the hippocampus, and stages V and VI are when the neurofibrillary tangles are extensive in the neocortical regions of the brain. A significant problem with Braak staging is that it can only be performed by post-mortem examination of the brain and thus cannot be used to prognose or diagnose living patients with Alzheimer's disease or Parkinson's disease.

One method used to diagnose Alzheimer's disease in living patients is Positron Emission Topography (PET), which can be used to highlight the presence of beta amyloid in the brain, whereby the build-up of beta amyloid protein in the brain is a hallmark feature of AD. However, PET scans are very costly, require the use of radioactive material and, while a lack of a positive scan can rule out Alzheimer's, many cognitively normal patients can have positive scans making interpretation, and therefore diagnosis, difficult.

There is therefore a need to provide improved methods and kits for prognosing and diagnosing patients with neurodegenerative disorders, and especially Alzheimer's disease.

The inventors have continued their previous work in this area, and have focused on the toxic peptide, T14, which is derived from the C-terminus of acetylcholinesterase (AChE), which is present as a naturally occurring bioactive molecule in brain tissue.

WO 2016/156803 describes an antibody raised against T14 which can selectively recognise and quantify this innate signalling molecule in either human and rat brain tissue or blood samples, by ELISA or Western blotting techniques.

The inventors have surprisingly shown that AChE-derived peptide (i.e. T14) levels closely correlate with Braak stages, such that measurements of T14 levels can be used to determine Braak stages in living subjects. Furthermore, the inventors were also surprised to observe that T14 levels can be used to distinguish between subjects that are amyloid positive and negative, and therefore to determine whether a PET scan is or is not required, thereby ensuring that expensive and highly invasive PET scans are not performed on subjects who do not need them, resulting in significant time and cost savings and less stress for the subject.

Accordingly, in a first aspect of the invention, there is provided a method of determining the Braak stage of a living subject, the method comprising:
(a) analysing, in a sample obtained from a living test subject, the concentration of (i) a soluble peptide comprising or consisting of SEQ ID No:3 (T14), or a variant or fragment thereof and/or (ii) an aggregated peptide comprising or consisting of SEQ ID No:3, or a variant or fragment thereof; and
(b) comparing this concentration with a reference value from a control population of deceased subjects having known Braak stages for concentrations of either soluble or aggregated peptide comprising or consisting of SEQ ID No:3, or a variant or fragment thereof,
wherein the Braak stage of the living test subject is determined by comparing the concentration of either the soluble or aggregated peptide comprising or consisting of SEQ ID No:3, or a variant or fragment thereof, with the respective reference value that is associated with a Braak stage.

In a second aspect, there is provided a Braak staging kit, for determining the Braak stage of a living subject, the kit comprising:
(a) means for determining, in a sample obtained from a test subject, the concentration of (i) a soluble peptide comprising or consisting of SEQ ID No:3, or a variant or fragment and/or (ii) an aggregated peptide comprising or consisting of SEQ ID No:3, or a variant or fragment thereof; and
(b) a reference value from a control population of deceased subjects having known Braak stages for concentrations of either soluble or aggregated peptide comprising or consisting of SEQ ID No:3, or a variant or fragment thereof,
wherein the kit is used to identify the Braak stage of the living subject by comparing the concentration of either soluble or aggregated peptide comprising or consisting of SEQ ID No:3, or a variant or fragment thereof, with the respective reference value that is associated with a Braak stage.

In a third aspect there is provided a method of treating a subject suffering from neurodegeneration and/or cognitive decline, the method comprising:
(a) analysing, in a sample obtained from a living subject, the concentration of (i) a soluble peptide comprising or consisting of SEQ ID No:3, or a variant or fragment thereof and/or (ii) an aggregated peptide comprising or consisting of SEQ ID No:3, or a variant or fragment thereof,
(b) determining the Braak stage of the subject by comparing the concentration of either soluble or aggregated peptide comprising or consisting of SEQ ID No:3, or a variant or fragment thereof, with a respective reference value from a control population of deceased subjects having known Braak stages for concentrations of either soluble or aggregated peptide comprising or consisting of SEQ ID No:3, or a variant or fragment thereof; and
(b) administering or having administered, to the subject, a therapeutic agent that prevents, reduces or delays neurodegeneration and/or cognitive decline.

It will be appreciated that the methods and kits of the invention may be used to determine and monitor disease progression in a method well-correlated with Braak stage. Advantageously, the results described in the Examples supports the inventors' hypothesis that detection of the peptide of SEQ ID No:3 (referred to herein as “T14”) in individuals can be used to determine the Braak stage of a living subject. Currently, Braak staging can only be performed on a post-mortem brain, and so the methods and kits of the invention provide a significant advance over these current methods. Use of the T14 biomarker (i.e. the peptide of SEQ ID No:3, or a variant or fragment thereof) allows the determination of the Braak stage of a patient with a very high degree of specificity and sensitivity from a non-invasive, easily repeatable, cost-effective procedure, such as blood, urine or CSF collection, and therefore allows routine screening, diagnosis of Braak stage and appropriate intervention with therapeutic treatment.

It will be appreciated that Braak staging has six stages based on the location of neurofibrillary tangles, with Braak stage 0 corresponding to a healthy subject. Stages I and II relates to early stage disease and is when neurofibrillary tangles are limited to the transentorhinal region of the brain. Stages III and IV define neurofibrillary tangle involvement in the limbic regions, which includes the hippocampus, and stages V and VI are when the neurofibrillary tangles are extensive in the neocortical regions of the brain. Accordingly, the methods and kits of the invention can be used to determine Braak stage 0, I, II, III, IV, V or VI of the living subject. Braak staging is a good method for recording progression of Parkinson's and Alzheimer's diseases in the post mortem brain, and is currently far more reliable than any ante-mortem method.

It will also be appreciated that the method according to the first aspect is useful for enabling a clinician to precisely diagnose the stage of neurodegeneration and/or cognitive decline, and therefore make informed decisions with regards to the best course of treatment for the patient based on their Braak stage. In addition, the method of the first aspect is useful for monitoring the efficacy of a putative treatment for neurodegeneration and cognitive decline. Hence, the kit according to the second aspect is useful for providing a prognosis of the subject's condition, such that the clinician can carry out the treatment according to the third aspect. The kit may also be used to monitor the efficacy of a putative treatment for neurodegeneration and cognitive decline. The method and the kit are therefore very useful for guiding a treatment regime for the clinician, and to monitor the efficacy of such a treatment regime.

Examples of suitable therapeutic agents which may be administered to the subject to prevent or treat the neurodegeneration and/or cognitive decline include, but are not limited to, acetylcholinesterase inhibitors, such as Rivastigmine, Galantamine, and Donepezil, and/or N-methyl-D-aspartate (NMDA) antagonists, such as Memantine.

Preferably a lower concentration of soluble peptide comprising or consisting of SEQ ID No:3, or a variant or fragment thereof, compared to the reference value is indicative of a later Braak stage. In other words, the lower the concentration of soluble T14, the greater the correlation with a later Braak stage, such as stage IV, V or VI, of the living subject.

Soluble peptide SEQ ID No:3 (T14), or a variant or fragment thereof, is preferably determined using ELISA, most preferably performed on a blood plasma sample taken from the subject.

Preferably, a higher concentration of soluble peptide comprising or consisting of SEQ ID No:3, or a variant or fragment thereof, compared to the reference value is indicative of a later Braak stage. In other words, the higher the concentration of soluble T14, the greater the correlation with a later Braak stage, such as stage IV, V or VI, of the living subject. Soluble peptide SEQ ID No:3 (T14), or a variant or fragment thereof, is preferably determined using ELISA, most preferably performed on a blood plasma sample taken from the subject.

Preferably, a higher concentration of aggregated peptide comprising or consisting of SEQ ID No:3, or a variant or fragment thereof, compared to the reference value is indicative of a later Braak stage. In other words, the higher the concentration of aggregated T14, the greater the correlation with a later Braak stage, such as stage IV, V or VI, of the living subject. Aggregated peptide SEQ ID No:3 (T14), or a variant or fragment thereof, is preferably determined using Western Blot, most preferably on a CSF sample taken from the subject.

Preferably, a lower concentration of aggregated peptide comprising or consisting of SEQ ID No:3, or a variant or fragment thereof, compared to the reference value is indicative of a later Braak stage. In other words, the lower the concentration of aggregated T14, the greater the correlation with a later Braak stage, such as stage IV, V or VI, of the living subject. Aggregated peptide SEQ ID No:3 (T14), or a variant or fragment thereof, is preferably determined using Western Blot, most preferably on a CSF sample taken from the subject.

The inventors believe that they are the first to describe the successful use of the T14 peptide as a biomarker for Braak staging living patients.

As such, in a fourth aspect of the invention, there is provided the use of a peptide comprising or consisting of SEQ ID No:3, or a variant or fragment thereof, as a biomarker for determining the Braak stage of a living subject.

In addition to measuring T14 concentrations to predict the Braak stage of a living patient, the inventors have also surprisingly found that concentrations of T14 can be used to determine if a subject requires a PET scan, as they have observed a good correlation between the two.

Accordingly, in a fifth aspect of the invention there is provided a method of determining if a subject should receive a positron emission tomography (PET) scan, the method comprising:
(a) analysing, in a sample obtained from a test subject, the concentration of (i) a soluble peptide comprising or consisting of SEQ ID No:3 (T14), or a variant or fragment thereof and/or (ii) an aggregated peptide comprising or consisting of SEQ ID No:3, or a variant or fragment thereof; and
(b) comparing this concentration with a reference value from a control population for concentrations of either a soluble or aggregated peptide comprising or consisting of SEQ ID No:3, or a variant or fragment thereof,
wherein a lower concentration of soluble peptide comprising or consisting of SEQ ID No:3, or a variant or fragment thereof, or an altered concentration of aggregated peptide comprising or consisting of SEQ ID No:3, or a variant or fragment thereof, compared to the respective reference value is indicative that the subject should receive a PET scan.

In another embodiment, the method of determining if a subject should receive a positron emission tomography (PET) scan, may comprise:
(a) analysing, in a sample obtained from a test subject, the concentration of (i) a soluble peptide comprising or consisting of SEQ ID No:3 (T14), or a variant or fragment thereof and/or (ii) an aggregated peptide comprising or consisting of SEQ ID No:3, or a variant or fragment thereof; and
(b) comparing this concentration with a reference value from a control population for concentrations of either a soluble or aggregated peptide comprising or consisting of SEQ ID No:3, or a variant or fragment thereof,
wherein a higher concentration of soluble peptide comprising or consisting of SEQ ID No:3, or a variant or fragment thereof, or an altered concentration of aggregated peptide comprising or consisting of SEQ ID No:3, or a variant or fragment thereof, compared to the respective reference value is indicative that the subject should receive a PET scan.

In a sixth aspect, there is provided a PET scan determining kit, for determining if a subject should receive a PET scan, the kit comprising:
(a) means for determining, in a sample obtained from a test subject, the concentration of (i) a soluble peptide comprising or consisting of SEQ ID No:3, or a variant or fragment and/or (ii) an aggregated peptide comprising or consisting of SEQ ID No:3, or a variant or fragment thereof; and
(b) a reference value from a control population for concentrations of soluble or aggregated peptide comprising or consisting of SEQ ID No:3, or a variant or fragment thereof,
wherein the kit is used to identify a lower concentration of soluble peptide comprising or consisting of SEQ ID No:3, and/or an altered concentration of aggregated peptide comprising or consisting of SEQ ID No:3, in the sample from the test subject, compared to the respective reference value, thereby suggesting that the subject should receive a PET scan.

Alternatively, the kit of the sixth aspect may comprise:
(a) means for determining, in a sample obtained from a test subject, the concentration of (i) a soluble peptide comprising or consisting of SEQ ID No:3, or a variant or fragment and/or (ii) an aggregated peptide comprising or consisting of SEQ ID No:3, or a variant or fragment thereof; and
(b) a reference value from a control population for concentrations of soluble or aggregated peptide comprising or consisting of SEQ ID No:3, or a variant or fragment thereof,
wherein the kit is used to identify a higher concentration of soluble peptide comprising or consisting of SEQ ID No:3, and/or an altered concentration of aggregated peptide comprising or consisting of SEQ ID No:3, in the sample from the test subject, compared to the respective reference value, thereby suggesting that the subject should receive a PET scan.

Soluble peptide SEQ ID No:3 (T14), or a variant or fragment thereof, is preferably determined using ELISA, preferably on a blood plasma sample taken from the subject. The inventors have surprisingly observed that a decreased concentration of T14 compared to the reference value corresponds to amyloid positive in the test subject, and that an increased concentration of T14 compared to the reference value corresponds to amyloid negative in the test subject.

Thus, preferably, a lower concentration of a soluble peptide comprising or consisting of

SEQ ID No:3 or a variant or fragment thereof compared to the reference value is indicative that the patient is beta amyloid positive, and/or a higher concentration of a soluble peptide comprising or consisting of SEQ ID No:3 or a variant or fragment thereof compared to the reference value is indicative that the patient is beta amyloid negative. Preferably, a lower concentration of a soluble peptide comprising or consisting of SEQ ID No:3 or a variant or fragment thereof compared to the reference value is indicative that the subject is cognitively impaired, and/or a higher concentration of a soluble peptide comprising or consisting of SEQ ID No:3 or a variant or fragment thereof compared to the reference value is indicative that the patient is cognitively normal.

Hence, in a preferred embodiment, a lower concentration of soluble peptide comprising or consisting of SEQ ID No:3, or a variant or fragment thereof, compared to the reference value is indicative of Braak stage IV. Alternatively, in another preferred embodiment, a lower concentration of soluble peptide comprising or consisting of SEQ ID No:3, or a variant or fragment thereof, compared to the reference value is indicative of Braak stage V. Alternatively, in another preferred embodiment, a lower concentration of soluble peptide comprising or consisting of SEQ ID No:3, or a variant or fragment thereof, compared to the reference value is indicative of Braak stage VI.

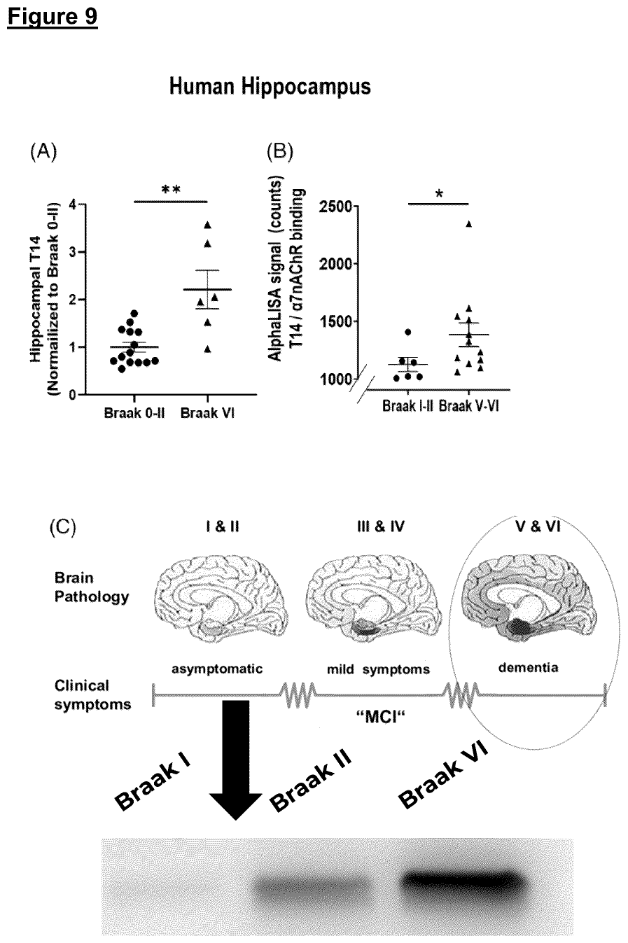

The inventors have also surprisingly discovered that the concentration of T14 differs amongst earlier Braak stages (see FIG. 9), therefore demonstrating that T14 levels can be used to determine the earlier pre-symptomatic Braak stages (I, II and III) in living patients. Accordingly, in one embodiment, a lower concentration of soluble peptide comprising or consisting of SEQ ID No:3, or a variant or fragment thereof, compared to the reference value is indicative of Braak stage I. Alternatively, in another embodiment, a lower concentration of soluble peptide comprising or consisting of SEQ ID No:3, or a variant or fragment thereof, compared to the reference value is indicative of Braak stage II. Alternatively, in another embodiment, a lower concentration of soluble peptide comprising or consisting of SEQ ID No:3, or a variant or fragment thereof, compared to the reference value is indicative of Braak stage III.

Hence, in one embodiment, a higher soluble T14 concentration compared to the reference value may correspond to amyloid negative status, wherein no PET scan is required. In another embodiment, a lower soluble T14 concentration compared to the reference value may correspond to amyloid positive status, wherein a PET scan is required.

In another embodiment, a higher concentration of a soluble peptide comprising or consisting of SEQ ID No:3 or a variant or fragment thereof compared to the reference value is indicative that the patient is beta amyloid positive, and/or a lower concentration of a soluble peptide comprising or consisting of SEQ ID No:3 or a variant or fragment thereof compared to the reference value is indicative that the patient is beta amyloid negative. Preferably, a higher concentration of a soluble peptide comprising or consisting of SEQ ID No:3 or a variant or fragment thereof compared to the reference value is indicative that the subject is cognitively impaired, and/or a lower concentration of a soluble peptide comprising or consisting of SEQ ID No:3 or a variant or fragment thereof compared to the reference value is indicative that the patient is cognitively normal.

Hence, in one embodiment, a lower soluble T14 concentration compared to the reference value may correspond to amyloid negative status, wherein no PET scan is required. In another embodiment, a higher soluble T14 concentration compared to the reference value may correspond to amyloid positive status, wherein a PET scan is required.

Aggregated peptide SEQ ID No:3 (T14), or a variant or fragment thereof, is preferably determined using Western Blot, more preferably on a CSF sample taken from the subject. The inventors have surprisingly observed that, in CSF, aggregated T14 is elevated in Alzheimer's patients compared to the reference value, and/or in plasma, aggregated T14 is lowered in Alzheimer's patients compared to the reference value. Furthermore, they have found that a decreased concentration of T14 compared to the reference value corresponds to pathology in the test subject, and/or that an increased concentration of T14 compared to the reference value corresponds to non-pathology in the test subject.

Hence, preferably, when the sample is CSF, a higher concentration of an aggregated peptide comprising or consisting of SEQ ID No:3 or a variant or fragment thereof compared to the reference value is indicative that the patient is beta amyloid positive, and/or a lower concentration of an aggregated peptide comprising or consisting of SEQ ID No:3 or a variant or fragment thereof compared to the reference value is indicative that the patient is beta amyloid negative. Preferably, when the sample is CSF, a higher concentration of an aggregated peptide comprising or consisting of SEQ ID No:3 or a variant or fragment thereof is indicative that the subject is cognitively impaired, and/or a lower concentration of an aggregated peptide comprising or consisting of SEQ ID No:3 or a variant or fragment thereof is indicative that the patient is cognitively normal.

Hence, in a preferred embodiment, when the sample is CSF, a higher concentration of aggregated peptide comprising or consisting of SEQ ID No:3, or a variant or fragment thereof, compared to the reference value is indicative of Braak stage IV. Alternatively, in another preferred embodiment, when the sample is CSF, a higher concentration of aggregated peptide comprising or consisting of SEQ ID No:3, or a variant or fragment thereof, compared to the reference value is indicative of Braak stage V. Alternatively, in another preferred embodiment, when the sample is CSF, a higher concentration of aggregated peptide comprising or consisting of SEQ ID No:3, or a variant or fragment thereof, compared to the reference value is indicative of Braak stage VI.

The inventors have also surprisingly discovered that the concentration of T14 differs amongst earlier Braak stages (see FIG. 9), therefore demonstrating that T14 levels can be used to determine the earlier pre-symptomatic Braak stages (I, II and III) in living patients. Accordingly, in one embodiment, when the sample is CSF, a higher concentration of aggregated peptide comprising or consisting of SEQ ID No:3, or a variant or fragment thereof, compared to the reference value is indicative of Braak stage I. Alternatively, in another embodiment, when the sample is CSF, a higher concentration of aggregated peptide comprising or consisting of SEQ ID No:3, or a variant or fragment thereof, compared to the reference value is indicative of Braak stage II. Alternatively, in another embodiment, when the sample is CSF, a higher concentration of aggregated peptide comprising or consisting of SEQ ID No:3, or a variant or fragment thereof, compared to the reference value is indicative of Braak stage III.

Preferably, when the sample is blood plasma, a lower concentration of an aggregated peptide comprising or consisting of SEQ ID No:3 or a variant or fragment thereof compared to the reference value is indicative that the patient is beta amyloid positive, and/or a higher concentration of an aggregated peptide comprising or consisting of SEQ ID No:3 or a variant or fragment thereof compared to the reference value is indicative that the patient is beta amyloid negative. Preferably, a lower concentration of an aggregated peptide comprising or consisting of SEQ ID No:3 or a variant or fragment thereof is indicative that the subject is cognitively impaired, and/or a higher concentration of an aggregated peptide comprising or consisting of SEQ ID No:3 or a variant or fragment thereof is indicative that the patient is cognitively normal.

Hence, in a preferred embodiment, when the sample is blood plasma, a lower concentration of aggregated peptide comprising or consisting of SEQ ID No:3, or a variant or fragment thereof, compared to the reference value is indicative of Braak stage IV. Alternatively, in another preferred embodiment, when the sample is blood plasma, a lower concentration of aggregated peptide comprising or consisting of SEQ ID No:3, or a variant or fragment thereof, compared to the reference value is indicative of Braak stage V. Alternatively, in another preferred embodiment, when the sample is blood plasma, a lower concentration of aggregated peptide comprising or consisting of SEQ ID No:3, or a variant or fragment thereof, compared to the reference value is indicative of Braak stage VI.

The inventors have also surprisingly discovered that the concentration of T14 differs amongst earlier Braak stages (see FIG. 9), therefore demonstrating that T14 levels can be used to determine the earlier pre-symptomatic Braak stages (I, II and III) in living patients. Accordingly, in one embodiment, when the sample is blood plasma, a lower concentration of aggregated peptide comprising or consisting of SEQ ID No:3, or a variant or fragment thereof, compared to the reference value is indicative of Braak stage I. Alternatively, in another embodiment, when the sample is blood plasma, a lower concentration of aggregated peptide comprising or consisting of SEQ ID No:3, or a variant or fragment thereof, compared to the reference value is indicative of Braak stage II. Alternatively, in another embodiment, when the sample is blood plasma, a lower concentration of aggregated peptide comprising or consisting of SEQ ID No:3, or a variant or fragment thereof, compared to the reference value is indicative of Braak stage III.

In another embodiment, when the sample is CSF, a lower concentration of an aggregated peptide comprising or consisting of SEQ ID No:3 or a variant or fragment thereof compared to the reference value is indicative that the patient is beta amyloid positive, and/or a higher concentration of an aggregated peptide comprising or consisting of SEQ ID No:3 or a variant or fragment thereof compared to the reference value is indicative that the patient is beta amyloid negative. Preferably, a lower concentration of an aggregated peptide comprising or consisting of SEQ ID No:3 or a variant or fragment thereof is indicative that the subject is cognitively impaired, and/or a higher concentration of an aggregated peptide comprising or consisting of SEQ ID No:3 or a variant or fragment thereof is indicative that the patient is cognitively normal.

Thus, preferably, when the sample is blood plasma, a higher concentration of an aggregated peptide comprising or consisting of SEQ ID No:3 or a variant or fragment thereof compared to the reference value is indicative that the patient is beta amyloid positive, and/or a lower concentration of an aggregated peptide comprising or consisting of SEQ ID No:3 or a variant or fragment thereof compared to the reference value is indicative that the patient is beta amyloid negative. Preferably, a higher concentration of an aggregated peptide comprising or consisting of SEQ ID No:3 or a variant or fragment thereof is indicative that the subject is cognitively impaired, and/or a lower concentration of an aggregated peptide comprising or consisting of SEQ ID No:3 or a variant or fragment thereof is indicative that the patient is cognitively normal.

The inventors believe that they are the first to describe the successful use of the T14 peptide as a biomarker for determining whether or not a subject should receive a PET scan. Given that PET scans are expensive, and require the use of radioactive material, they should be avoided if at all possible unless the clinician suspects that they would provide important information regarding the patient. Although a lack of a positive scan can rule out Alzheimer's, many cognitively normal patients can have positive scans making interpretation, and therefore diagnosis, difficult.

Accordingly, in a seventh aspect, there is provided the use of a peptide comprising or consisting of SEQ ID No:3, or a variant or fragment thereof, as a biomarker for determining if a subject requires a PET scan.

The invention may be used in a method or kit for determining the Braak stage of a living subject, or determine if a PET scan is required, where the subject has, or is suspected of having, a neurodegenerative disease selected from a group consisting of: Alzheimer's disease; Parkinson's disease; Huntington's disease; Motor Neurone disease; Spinocerebellar type 1, type 2, and type 3; Amyotrophic Lateral Sclerosis (ALS); schizophrenia; Lewy-body dementia; and Frontotemporal Dementia. It is preferred, however, that the invention is used to study or predict cognitive decline in any neurological disorder associated with non-enzymatic function of AChE, in particular, for example, Alzheimer's Disease, Parkinson's Disease and Motor Neuron Disease, and preferably Alzheimer's Disease and Parkinson's Disease.

However, it is especially preferred that the methods and kits of invention are used to determine the Braak stage of a living subject, or determine if the subject requires a PET scan, where the subject has, or is suspected of having, Alzheimer's Disease.

Following on from their previous studies, the inventors continued their research with acetylcholinesterase, and their antibody which exhibits immunospecificity to specific regions in the C-terminus of this enzyme, as described in WO 2016/156803, the contents of which are incorporated herein by reference.

Acetylcholinesterase is a serine protease that hydrolyses acetylcholine, and is well-known to the skilled person. The major form of acetylcholinesterase which is found in the brain is known as tailed acetylcholinesterase (T-AChE). The protein sequence of one embodiment of human tailed acetylcholinesterase (Gen Bank: AAA68151.1) is 614 amino acids in length, and is provided herein as SEQ ID No:1, as follows:

| [SEQ ID No: 1] | |
|---|---|
| 1 | mrppqcllht pslaspllll llwllgggvg aegredaell vtvrggrlrg irlktpggpv |
| 61 | saflgipfae ppmgprrflp pepkqpwsgv vdattfqsvc yqyvdtlypg fegtemwnpn |
| 121 | relsedclyl nvwtpyprpt sptpvlvwiy gggfysgass ldvydgrflv qaertvlvsm |
| 181 | nyrvgafgfl alpgsreapg nvglldqrla lqwvqenvaa fggdptsvtl fgesagaasv |
| 241 | gmhllsppsr glfhravlqs gapngpwatv gmgearrrat qlahlvgcpp ggtggndtel |
| 301 | vaclrtrpaq vlvnhewhvl pqesvfrfsf vpvvdgdfls dtpealinag dfhglqvlvg |
| 361 | vvkdegsyfl vygapgfskd neslisraef lagvrvgvpq vsdlaaeavv lhytdwlhpe |
| 421 | dparlreals dvvgdhnvvc pvaqlagrla aqgarvyayv fehrastlsw plwmgvphgy |
| 481 | eiefifgipl dpsrnytaee kifaqrlmry wanfartgdp neprdpkapq wppytagaqq |
| 541 | yvsldlrple vrrglraqac afwnrflpkl lsatdtldea erqwkaefhr wssymvhwkn |
| 601 | qfdhyskqdr csdl |

The amino acid sequence of T30 (which corresponds to the last 30 amino acid residues of SEQ ID No:1) is provided herein as SEQ ID No:2, as follows:

[SEQ ID No: 2]
KAEFHRWSSYMVHWKNQFDHYSKQDRCSDL

The amino acid sequence of T14 (which corresponds to the 14 amino acid residues located towards the end of SEQ ID No:1, and lacks the final 15 amino acids found in T30) is provided herein as SEQ ID No:3, as follows:

[SEQ ID No: 3]
AEFHRWSSYMVHWK

Preferably, therefore, the peptide of SEQ ID No:3, or a variant or fragment thereof is T14. Most preferably, the methods and kits of the invention involve detection of soluble and/or aggregated peptide comprising or consisting of SEQ ID No:3.

However, fragments of T14 (SEQ ID No:3) are also detectable in the methods and kits of the invention, and can act as diagnostic or prognostic markers used in accordance with the invention.

Thus, in one embodiment, a fragment of SEQ ID No:3 preferably comprises an amino acid sequence of SEQ ID No:4 (i.e. T7), i.e. SYMVHWK.

In another embodiment, a fragment of SEQ ID No:3 preferably comprises an amino acid sequence of SEQ ID No: 5 (i.e. T8), i.e. SSYMVHWK.

In another embodiment, a fragment of SEQ ID No:3 preferably comprises an amino acid sequence of SEQ ID No: 6 (i.e. T9), i.e. WSSYMVHWK.

In another embodiment, a fragment of SEQ ID No:3 preferably comprises an amino acid sequence of SEQ ID No: 7 (i.e. T10), i.e. RWSSYMVHWK.

In another embodiment, a fragment of SEQ ID No:3 preferably comprises an amino acid sequence of SEQ ID No: 8 (i.e. T11), i.e. HRWSSYMVHWK.

In another embodiment, a fragment of SEQ ID No:3 preferably comprises an amino acid sequence of SEQ ID No: 9 (i.e. T12), i.e. FHRWSSYMVHWK.

In another embodiment, a fragment of SEQ ID No:3 preferably comprises an amino acid sequence of SEQ ID No:10 (i.e. T13), i.e. EFHRWSSYMVHWK.

In other words, although detection of T14 (i.e. SEQ ID No:3) is preferred, the invention may also rely on detection of one or more of any of T7-T13 (i.e. SEQ ID No: 4-10).

The subject may be a vertebrate, mammal, or domestic animal. Most preferably, however, the subject is a human being, who may be male or female. The subject may be a child or adult. The age of the subject may be at least 30, 40, 50, 60 or 70 years old. The subject, however, may be less than 80, 70, or 60 years old. Preferably, the subject is tested before any symptoms of neurodegeneration, cognitive decline or neurodegenerative disorder are apparent. It is envisaged that the invention may be carried out at a similar time and in a similar manner to the way body mass index (BMI) measurements are taken.

Preferably, the method is carried out in vitro. The kit may comprise a sample collection container for receiving the extracted sample. Preferably, the sample comprises a biological sample. The sample may be any material that is obtainable from the subject from which protein is obtainable. Furthermore, the sample may be blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, or combinations thereof.

Preferably, however, the sample comprises blood, urine, tissue, CSF etc. Most preferably, the sample comprises a blood sample. The blood may comprise venous or arterial blood.

Blood samples may be assayed for T14 levels immediately. Alternatively, the blood sample may be stored at low temperatures, for example in a fridge or even frozen before the T14 assay is conducted. Detection of T14 may be carried out on whole blood. Preferably, however, the blood sample comprises blood serum. Preferably, the blood sample comprises blood plasma.

The blood may be further processed before the T14 assay is performed. For instance, an anticoagulant, such as citrate (such as sodium citrate), hirudin, heparin, PPACK, or sodium fluoride may be added. Thus, the sample collection container may contain an anticoagulant in order to prevent the blood sample from clotting. Alternatively, the blood sample may be centrifuged or filtered to prepare a plasma or serum fraction, which may be used for analysis. Hence, it is preferred that the T14 is analysed or assayed in a blood plasma or a blood serum sample. It is especially preferred that T14 concentration is measured in vitro from a blood serum sample or a plasma sample taken from the subject.

Preferably, the kit or method is used to identify the presence or absence of T14-positive cells (i.e. cells comprising SEQ ID No:3) in the sample, or determine the concentration thereof in the sample, preferably the concentration of soluble T14 and/or aggregated T14. The means for determining the T14 concentration, be it soluble or aggregated T14, may comprise an assay adapted to detect the presence and/or absence of T14-positive cells in the sample. The kit or method may comprise the use of a positive control and/or a negative control against which the assay may be compared.

T14 peptide (SEQ ID No:3) may be assayed by a number of ways known to one skilled in the art. For example, preferably an immunoassay is employed to measure T14 peptide levels. However, it will be appreciated that non-immuno based assays may be employed, for example, labelling a compound having affinity with a ligand of the T14 peptide molecule, and then assaying for the label. T14 peptide may also be determined with Western Blot analysis, which may be used to determine the total protein level of T14 peptide. T14 peptide concentration may therefore be detected by enzyme-linked immunosorbent assay (ELISA), fluorometric assay, chemiluminescent assay, or radioimmunoassay analyses.

An immunoassay, such as ELISA, is most preferably used to detect soluble T14 peptide. Western Blot analysis is most preferably used to detect aggregated T14 peptide. It is especially preferred that the methods and kits and uses of the invention comprise detection of soluble T14 (SEQ ID No:3).

In one embodiment of the invention, the concentration of (i) a soluble peptide comprising or consisting of SEQ ID No:3, or a variant or fragment thereof, or of (ii) an aggregated peptide comprising or consisting of SEQ ID No:3, or a variant or fragment thereof, is determined. In a preferred embodiment, however, the concentration of (i) a soluble peptide comprising or consisting of SEQ ID No:3, or a variant or fragment thereof, and of (ii) an aggregated peptide comprising or consisting of SEQ ID No:3, or a variant or fragment thereof, is determined. It is preferred that both soluble and aggregated T14 can be detected in combination. Although the inventors do not wish to be bound by hypothesis, they have observed a 2 kDa band and also a 50 kDa band on their Western blots. Their aggregation experiments do not account for the difference in molecular weight, because a change in molecular weight was not observed when triggering aggregation in laboratory conditions. The inventors believe that that T14 peptide could be bound to a higher molecular weight protein, and that this interaction is not disrupted by the denaturing and reducing conditions of the Western Blot.

The means for determining, in the sample obtained from the test subject, the concentration of (i) a soluble T14 and/or (ii) an aggregated T14 may comprise an anti-T14 antibody or antigen-binding fragment thereof, i.e. a T14-neutralising antibody. The antibody or antigen-binding fragment thereof may be polyclonal or monoclonal. The antibody or antigen-binding fragment thereof may be generated in a rabbit, mouse or rat.

Preferably, the antibody or antigen-binding fragment thereof specifically binds to SEQ ID No:3. Preferably, the antibody or antigen-binding fragment thereof specifically binds to one or more amino acid in the C-terminus of SEQ ID No:3. Preferably, the antibody or antigen-binding fragment thereof specifically binds to one or more amino acid in SEQ ID No: 11 (i.e. SYMVHWK, which are the C-terminal amino acids numbers 7-14 of SEQ ID No: 3). Preferably, the antibody or antigen-binding fragment thereof specifically binds to a C-terminal lysine (K) residue in the epitope. The inventors have surprisingly observed that the C-terminal amino acid sequence VHWK in SEQ ID No:3, which is described herein as SEQ ID No. 12 (i.e. the C-terminal amino acids numbers 11-14 of SEQ ID No.3), acts as an epitope for the antibody or antigen-binding fragment thereof. Accordingly, more preferably the antibody or antigen-binding fragment thereof specifically binds to one or more amino acid in SEQ ID No.12. Most preferably, the antibody or antigen-binding fragment thereof specifically binds to SEQ ID No. 12. Hence, it will be appreciated that the epitope to which the antibody binds comprises or consists of SEQ ID No: 12. Thus, the antibody or antigen-binding fragment thereof binds specifically to SEQ ID No:3, or a fragment or variant thereof, and can be used as or in the detection means.

Preferably, the antibody or antigen-binding fragment thereof does not bind to SEQ ID No:2 (i.e. T30).

Preferably, the antibody or antigen-binding fragment thereof does not bind to SEQ ID No:13 (i.e. T15), i.e. NQFDHYSKQDRCSDL.

Preferably, the antibody or antigen-binding fragment thereof does not bind to SEQ ID No: 14 (i.e. β-amyloid (Aβ), i.e.

### DAEFRHDSGYEVHHQKLVFFAEDVGSNKGAIIGLMVGGVVIA.

The kits of the second or sixth aspects may further comprise a label which may be detected. The term “label” can mean a moiety that can be attached to the means for determining, in the sample obtained from the test subject, the concentration of (i) a soluble T14 and/or (ii) an aggregated T14. Moieties can be used, for example, for therapeutic or diagnostic procedures. Therapeutic labels include, for example, moieties that can be attached to the antibody or fragment thereof described herein, and used to monitor the binding of the antibody to the T14 peptide (i.e. SEQ ID No:3, or fragment or variant thereof). Diagnostic labels include, for example, moieties which can be detected by analytical methods. Analytical methods include, for example, qualitative and quantitative procedures. Qualitative analytical methods include, for example, immunohistochemistry and indirect immunofluorescence. Quantitative analytical methods include, for example, immunoaffinity procedures such as radioimmunoassay, ELISA or FACS analysis. Analytical methods also include both in vitro and in vivo imaging procedures. Specific examples of diagnostic labels that can be detected by analytical means include enzymes, radioisotopes, fluorochromes, chemiluminescent markers, and biotin.

Preferably, soluble T14 peptide serum or T14 peptide plasma concentrations may be measured by double-antibody sandwich ELISA, which will be known to the skilled technician. The ELISA may comprise using a suitable antibody for coating a microtiter plate. For example, such a suitable antibody may comprise an affinity-purified polyclonal anti-T14 peptide antibody described herein (WO 2016/156803).

Furthermore, the ELISA may comprise using a suitable antibody for detection. For example, such a suitable antibody may comprise peroxidase-labelled monoclonal mouse anti human T14 peptide antibody. Human T14 peptide, which may be purified from plasma, and which then may be quantified by amino acid analysis, may be used to calibrate a plasma standard using standard techniques known to the skilled technician. A label can be attached directly to the antibody, or be attached to the secondary binding agent that specifically binds T14. Such a secondary binding agent can be, for example, a secondary antibody. A secondary antibody can be either polyclonal or monoclonal, and of human, rodent or chimeric origin.

The skilled technician will appreciate how to measure the concentrations of T14 peptide (either soluble or aggregated) in a statistically significant number of control individuals, and the concentration of T14 in the test subject, and then use these respective figures to determine the Braak stage of the test subject, or to determine whether the subject is in need of a PET scan. Comparing the levels of the peptide of SEQ ID No: 3 (i.e. T14) in a sample (preferably, blood plasma) collected from a large group of well-characterised Braak stage 0 individuals (i.e. no disease or “normal” health) as close as possible to subject death may be a preferred method of defining a control population (i.e. cohort) of the reference value.

In another embodiment, after collection of post-mortem CSF samples from a sufficient number of control and Alzheimer's Disease subjects (e.g. n>50 for each group), their soluble and aggregated T14 levels may be measured by ELISA and Western Blot, respectively. These levels are preferably calibrated to the subject's Braak stages (=0 for controls) to determine the relationship between the change in T14 and incremental increase in Braak staging. The resultant standard curve can be used by future ex-vivo CSF samples from living patients to extrapolate their Braak stage from their T14 levels in the patient's asymptomatic stage of disease.

In another embodiment, for blood plasma, samples may be required from a sufficient number of control and AD subjects (e.g. n>200 for each group). Their soluble and aggregated T14 levels may be measured by ELISA and Western Blot, respectively. These levels may be normalised to that from a healthy subject. This normalisation step may not be needed. Next, the range of control and AD values may be plotted with confidence intervals. Single samples from patients with no disease symptoms may be diagnosed or disease progression predicted by detecting whether or not T14 values falling within or outside of control T14 ranges or falling within or outside AD T14 ranges.

Accordingly, the inventors have realised that the difference in concentrations of T14 between the normal and raised/lowered levels, for aggregated or soluble T14, respectively, can be used as a physiological marker, to determine the Braak stage of a living subject and to determine whether a subject is in need of a PET scan. It will be appreciated that if a subject has a lowered concentration of soluble T14 which is considerably lower than the reference soluble T14 concentration, or a raised concentration of aggregated T14 which considerably higher than the reference aggregated T14 concentration, then this would be indicative of a higher Braak stage and/or that the subject requires a PET scan. The inventors have also discovered that the concentration of T14 differs amongst earlier Braak stages, therefore demonstrating that T14 levels can be used to determine the earlier pre-symptomatic Braak stages (I, II and III) in living patients.

By way of example, the decrease in concentration of soluble T14 from the reference concentration may be at least 10%, preferably at least a 20% decrease, more preferably at least a 30% decrease, even more preferably at least a 40% decrease, and most preferably a decrease of at least 50% from the reference value concentration. Such decreases in soluble T14 concentrations infer that the test subject will have a higher Braak stage and/or require a PET scan. Alternatively, the increase in concentration of aggregated T14 from the reference concentration may be approximately at least 10%, preferably about at least a 20% increase, more preferably at least a 30% increase, even more preferably at least a 40% increase, and most preferably an increase of at least 50% from the reference value concentration. Such increases in aggregated T14 concentrations infer that the test subject will have a higher Braak stage and/or require a PET scan. Accordingly, a clinician would be able to make an informed decision as to the preferred course of treatment required, for example, the type and dosage of the therapeutic agent according to the third aspect to be administered and whether or not the subject should undergo a PET scan.

In another embodiment, it will be appreciated that if a subject has a raised concentration of soluble T14 which is considerably higher than the reference soluble T14 concentration, or a lower concentration of aggregated T14 which considerably lower than the reference aggregated T14 concentration, then this would be indicative of a lower Braak stage and/or that the subject requires a PET scan.

By way of example, the increase in concentration of soluble T14 from the reference concentration may be at least 10%, preferably at least a 20% increase, more preferably at least a 30% increase, even more preferably at least a 40% increase, and most preferably a increase of at least 50% from the reference value concentration. Such increase in soluble T14 concentrations infer that the test subject will have a higher Braak stage and/or require a PET scan. Alternatively, the decrease in concentration of aggregated T14 from the reference concentration may be approximately at least 10%, preferably about at least a 20% decrease, more preferably at least a 30% decrease, even more preferably at least a 40% decrease, and most preferably an decrease of at least 50% from the reference value concentration. Such decreases in aggregated T14 concentrations infer that the test subject will have a higher Braak stage and/or require a PET scan. Accordingly, a clinician would be able to make an informed decision as to the preferred course of treatment required, for example, the type and dosage of the therapeutic agent according to the third aspect to be administered and whether or not the subject should undergo a PET scan.

The methods of the invention may further comprise measuring the rate of cognitive decline by a Mini Mental State Examination (MMSE) score and/or a Preclinical Alzheimer Cognitive Composite (PACC) score.

MMSE is a questionnaire that is administered virtually universally in those with suspected AD, and in broader study cohorts, as a measure of cognitive impairment. It is widely considered a gold standard for diagnosis in AD due to its ease of application with little training required, repeatability, validity and reliability. It is also particularly useful when considering the longitudinal assessment of AD and its progression. Repeated measures of MMSE score, collected at regular intervals, may be used to calculate a rate of cognitive decline for the subject. Then, a linear regression is preferably performed to calculate the slope of MMSE score change over time, and it is this slope which is interpreted as the rate of cognitive change (decline/incline). Preferably, therefore, cognitive decline is measured in terms of MMSE score. The slope may be calculated in points dropped on the MMSE score per month.

The PACC test, on the other hand, combines tests that assess episodic memory, timed executive function, and global cognition. It is the primary outcome measure for the first clinical trial in preclinical AD.

The methods of the invention may further comprise a step of age-adjusting the T14 concentrations from the test subject, be they soluble T14 or aggregated T14, against the corresponding reference value.

It will be appreciated that the invention extends to any nucleic acid or peptide or variant, derivative or analogue thereof, which comprises substantially the amino acid or nucleic acid sequences of any of the sequences referred to herein, including variants or fragments thereof. The terms “substantially the amino acid/nucleotide/peptide sequence”, “variant” and “fragment”, can be a sequence that has at least 40% sequence identity with the amino acid/nucleotide/peptide sequences of any one of the sequences referred to herein, for example 40% identity with the sequence identified as SEQ ID Nos: 1-14, and so on.

Amino acid/polynucleotide/polypeptide sequences with a sequence identity which is greater than 65%, more preferably greater than 70%, even more preferably greater than 75%, and still more preferably greater than 80% sequence identity to any of the sequences referred to are also envisaged. Preferably, the amino acid/polynucleotide/polypeptide sequence has at least 85% identity with any of the sequences referred to, more preferably at least 90% identity, even more preferably at least 92% identity, even more preferably at least 95% identity, even more preferably at least 97% identity, even more preferably at least 98% identity and, most preferably at least 99% identity with any of the sequences referred to herein.

The skilled technician will appreciate how to calculate the percentage identity between two amino acid/polynucleotide/polypeptide sequences. In order to calculate the percentage identity between two amino acid/polynucleotide/polypeptide sequences, an alignment of the two sequences must first be prepared, followed by calculation of the sequence identity value. The percentage identity for two sequences may take different values depending on:—(i) the method used to align the sequences, for example, ClustalW, BLAST, FASTA, Smith-Waterman (implemented in different programs), or structural alignment from 3D comparison; and (ii) the parameters used by the alignment method, for example, local vs global alignment, the pair-score matrix used (e.g. BLOSUM62, PAM250, Gonnet etc.), and gap-penalty, e.g. functional form and constants.

Having made the alignment, there are many different ways of calculating percentage identity between the two sequences. For example, one may divide the number of identities by: (i) the length of shortest sequence; (ii) the length of alignment; (iii) the mean length of sequence; (iv) the number of non-gap positions; or (v) the number of equivalenced positions excluding overhangs. Furthermore, it will be appreciated that percentage identity is also strongly length dependent. Therefore, the shorter a pair of sequences is, the higher the sequence identity one may expect to occur by chance.

Hence, it will be appreciated that the accurate alignment of protein or DNA sequences is a complex process. The popular multiple alignment program ClustalW (Thompson et al., 1994, Nucleic Acids Research, 22, 4673-4680; Thompson et al., 1997, Nucleic Acids Research, 24, 4876-4882) is a preferred way for generating multiple alignments of proteins or DNA in accordance with the invention. Suitable parameters for ClustalW may be as follows: For DNA alignments: Gap Open Penalty=15.0, Gap Extension Penalty=6.66, and Matrix=Identity. For protein alignments: Gap Open Penalty=10.0, Gap Extension Penalty=0.2, and Matrix=Gonnet. For DNA and Protein alignments: ENDGAP=−1, and GAPDIST=4. Those skilled in the art will be aware that it may be necessary to vary these and other parameters for optimal sequence alignment.

Preferably, calculation of percentage identities between two amino acid/polynucleotide/polypeptide sequences may then be calculated from such an alignment as (N/T)*100, where N is the number of positions at which the sequences share an identical residue, and T is the total number of positions compared including gaps and either including or excluding overhangs. Preferably, overhangs are included in the calculation. Hence, a most preferred method for calculating percentage identity between two sequences comprises (i) preparing a sequence alignment using the ClustalW program using a suitable set of parameters, for example, as set out above; and (ii) inserting the values of N and T into the following formula:—Sequence Identity=(N/T)*100.

Alternative methods for identifying similar sequences will be known to those skilled in the art. For example, a substantially similar nucleotide sequence will be encoded by a sequence which hybridizes to DNA sequences or their complements under stringent conditions. By stringent conditions, the inventors mean the nucleotide hybridises to filter-bound DNA or RNA in 3× sodium chloride/sodium citrate (SSC) at approximately 45° C. followed by at least one wash in 0.2×SSC/0.1% SDS at approximately 20-65° C. Alternatively, a substantially similar polypeptide may differ by at least 1, but less than 5, 10, 20, 50 or 100 amino acids from the sequences shown in, for example, SEQ ID Nos: 1-14.

Due to the degeneracy of the genetic code, it is clear that any nucleic acid sequence described herein could be varied or changed without substantially affecting the sequence of the protein encoded thereby, to provide a functional variant thereof. Suitable nucleotide variants are those having a sequence altered by the substitution of different codons that encode the same amino acid within the sequence, thus producing a silent (synonymous) change. Other suitable variants are those having homologous nucleotide sequences but comprising all, or portions of, sequence, which are altered by the substitution of different codons that encode an amino acid with a side chain of similar biophysical properties to the amino acid it substitutes, to produce a conservative change. For example small non-polar, hydrophobic amino acids include glycine, alanine, leucine, isoleucine, valine, proline, and methionine. Large non-polar, hydrophobic amino acids include phenylalanine, tryptophan and tyrosine. The polar neutral amino acids include serine, threonine, cysteine, asparagine and glutamine. The positively charged (basic) amino acids include lysine, arginine and histidine. The negatively charged (acidic) amino acids include aspartic acid and glutamic acid. It will therefore be appreciated which amino acids may be replaced with an amino acid having similar biophysical properties, and the skilled technician will know the nucleotide sequences encoding these amino acids.

All of the features described herein (including any accompanying claims, abstract and drawings), and/or all of the steps of any method or process so disclosed, may be combined with any of the above aspects in any combination, except combinations where at least some of such features and/or steps are mutually exclusive.

For a better understanding of the invention, and to show how embodiments of the same may be carried into effect, reference will now be made, by way of example, to the accompanying Figures, in which:

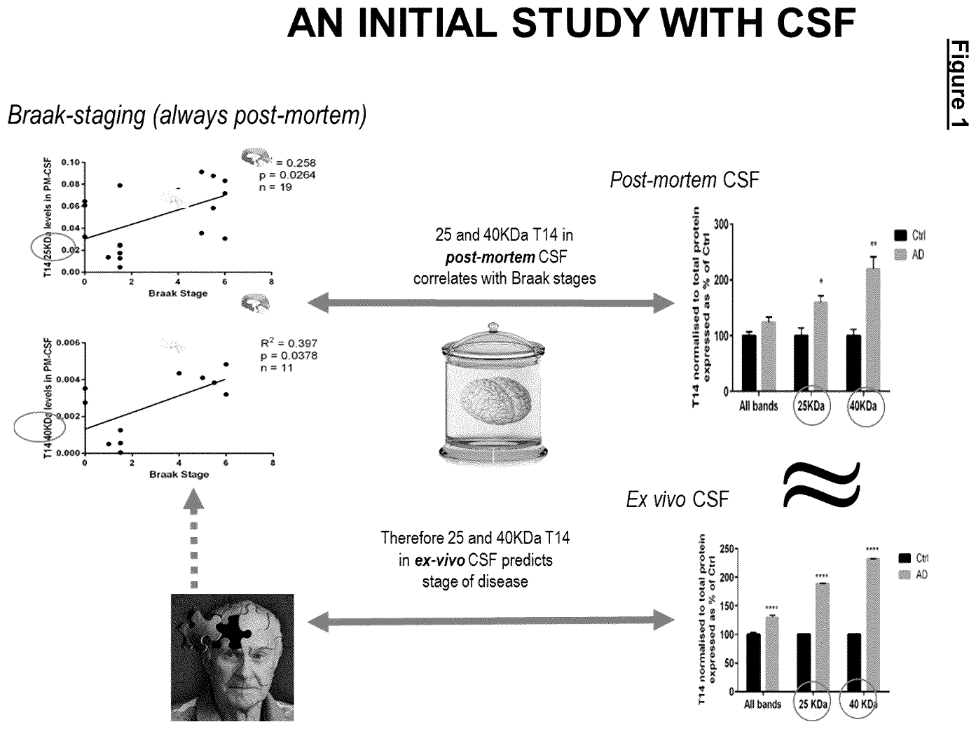

FIG. 1 shows how the T14 western blot profile in Alzheimer CSF can, for the first time, indicate the degree of neurodegeneration in the brains of living patients. Post-mortem Braak staging is significantly correlated with the increase in T14 binding at two weights, 25 KDa and 40 KDa. These same two bands are also significantly increased in the CSF of living patients, acting as a means of extrapolating the corresponding stage of brain pathology during the course of an individual's disease.

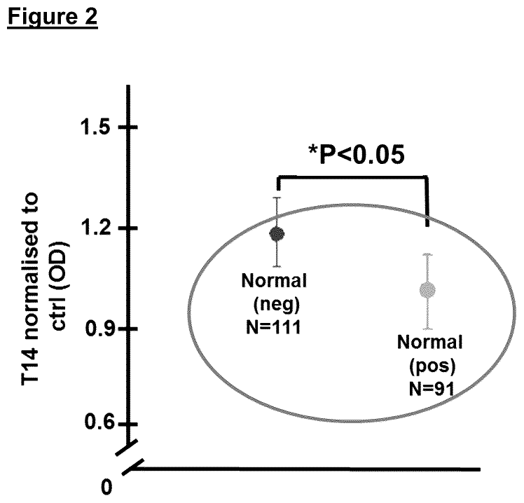

FIG. 2 shows the levels of T14 detected by ELISA (Adjusted mean with 95% confidence limits). T14 is able to clearly differentiate between amyloid positive and negative with significance levels *<0.05.

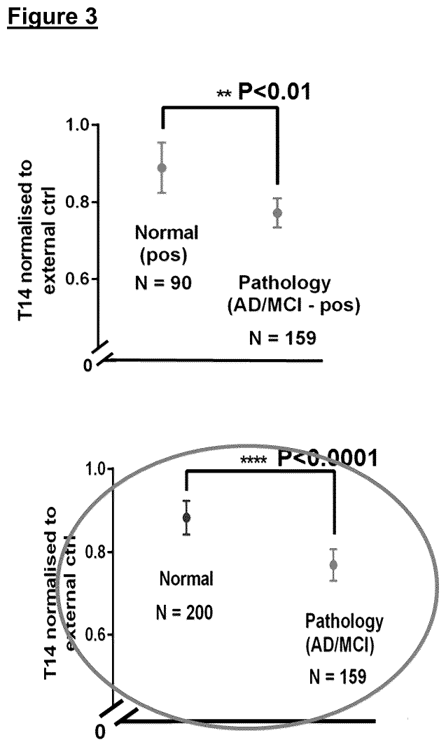

FIG. 3 shows levels of T14 detected by Western Blot (Adjusted mean with 95% confidence limits). On the right, T14 is able to differentiate between normal and pathology with significance levels ****<0.0001, and between normal amyloid positive and pathology amyloid positive subjects ** <0.01 (left).

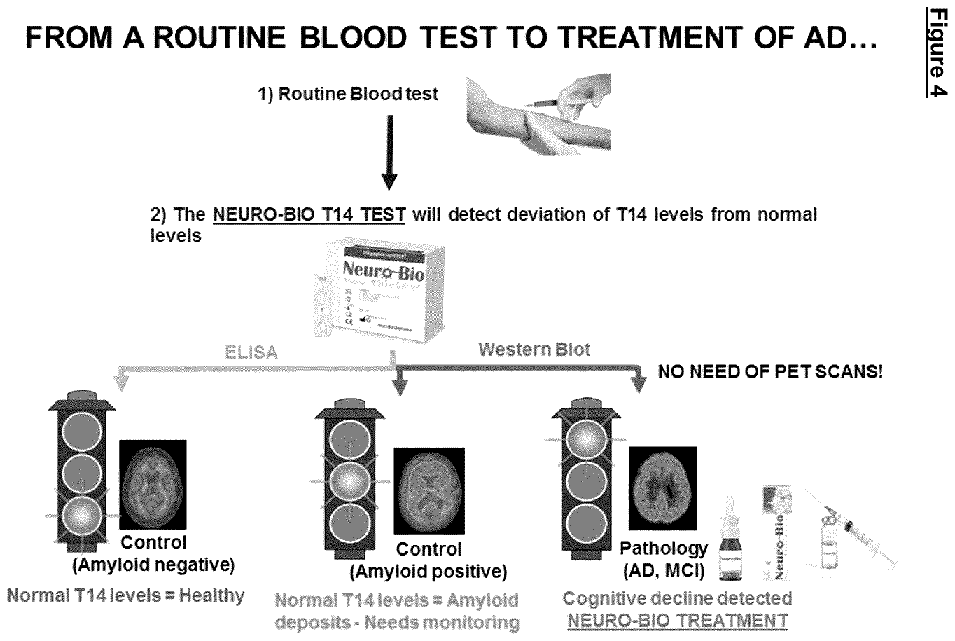

FIG. 4 shows a scheme for allocation of subjects into 1 of 3 groups (normal amyloid negative, normal amyloid positive and pathology) based on a single blood test but using the 2 complementary T14 tests.

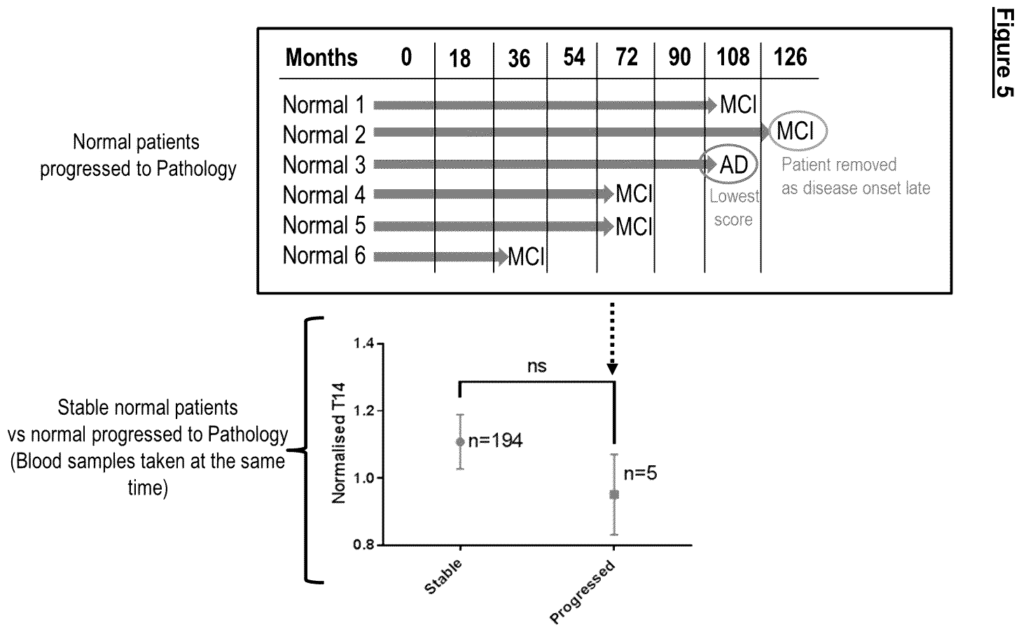

FIG. 5 shows, in the upper figure, the progress of six subjects over the months that have converted to pathological status. The lower figure shows the levels of T14 in plasma divided into two groups, all cognitively normal at the time of sampling: normal subjects remaining stable (n=194) and subjects normal at the time of sampling but progressing to pathology (n=5, subjects normal 1 to 6).

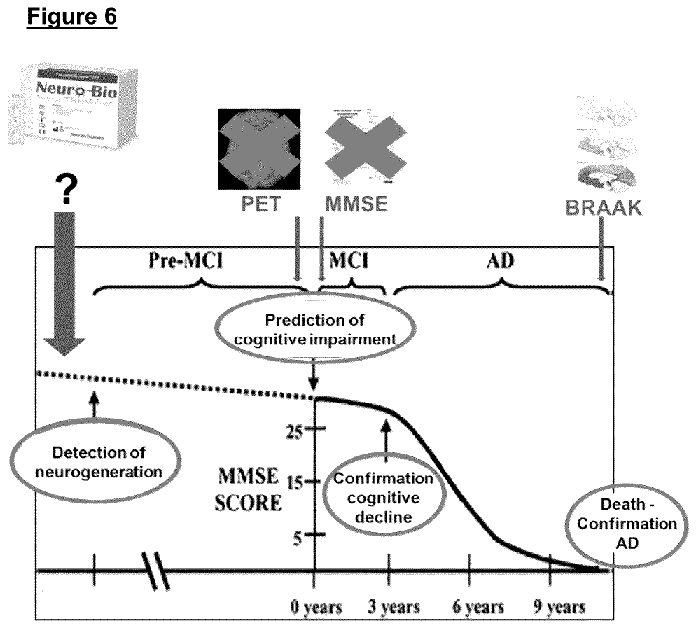

FIG. 6 shows a scheme differentiating the onset of neurodegeneration from the subsequent cognitive impairment as measured by MMSE and PET scans. The T14 biomarker has the potential for detecting the neurodegeneration process itself rather than the much later onset of cognitive decline monitored currently by the conventional tests.

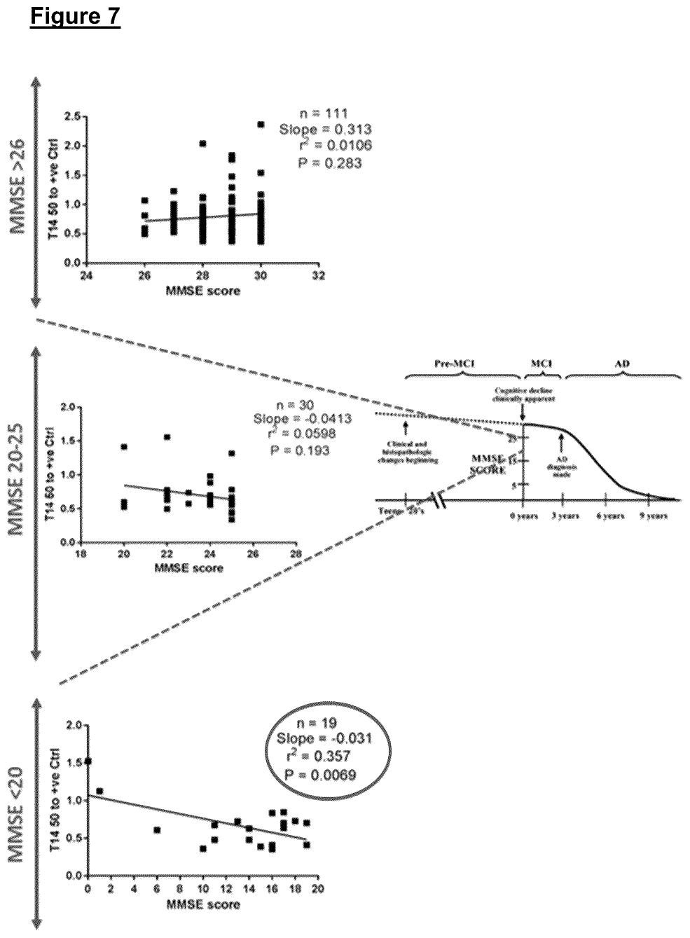

FIG. 7 shows the levels of T14 detected by Western Blot in single samples correlated to the MMSE at the time when the blood was taken (3 groups: >26, 20-25 and <20). Y axis: Level of T14 50 KDa band normalised to positive control in the blood plasma sample. The blood sample was taken a period of time after the initial diagnosis of AD. X axis: MMSE score at the time when the blood sample was taken, a period of time after the initial diagnosis.

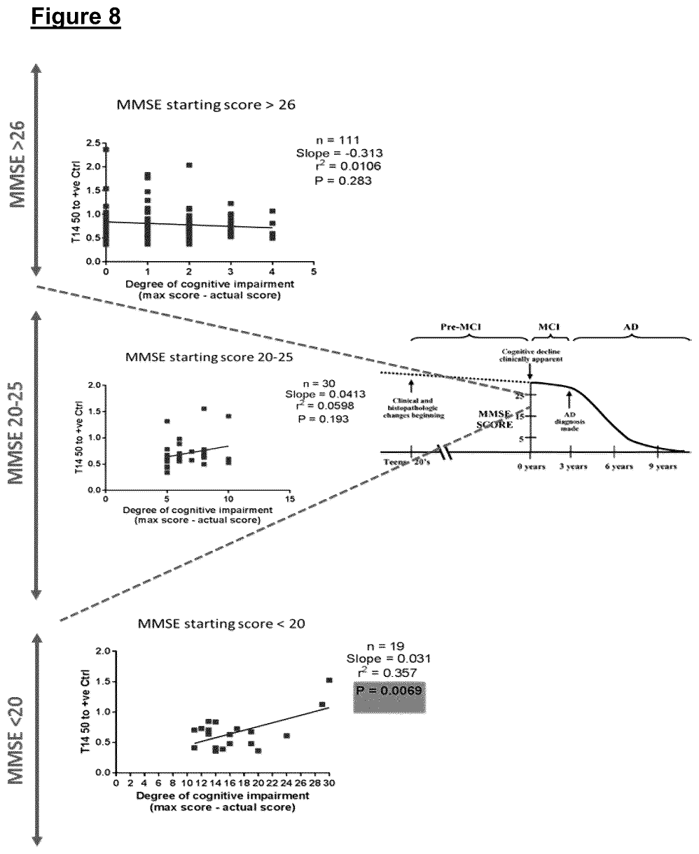

FIG. 8 shows T14 levels detected by Western Blot in single samples correlated with the degree of cognitive impairment based on basal MMSE score. Y axis: Level of T14 50 KDa band normalised to positive control in the blood plasma sample. The blood sample was taken at the final time point of consecutive assessment after the initial diagnosis of AD. X axis: ‘Degree of cognitive impairment’=MMSE at the time of initial AD diagnosis−MMSE at the time when the blood sample was taken.

FIG. 9 shows (A) quantification of hippocampal T14 (mean±SEM) normalised to the data for Braak stage 0-II (n=14), showing a significant increase at Braak stage VI (n=6); (B) T14-alpha-7 receptor binding using AlphaLISA in early stage (Braak I-II, n=6) and late stage (Braak V-VI, n=12) hippocampal tissue; and (C) western blots for hippocampal T14 at Braak stage I, II and VI, showing an increase at the later stage.

### EXAMPLES

### Materials and Methods

### Human Clinical Samples

Post-mortem CSF samples were supplied by the Thomas Willis Oxford Brain Collection. An ethics application was approved by the Human Tissue Bank of the Oxford Radcliffe Hospital NHS, complying with the Human Tissue Act, Human Tissue Authority Codes of Practice and other laws relevant to post-mortem examinations and use of tissues. Ex-vivo CSF from four AD cases and three age-matched controls was provided by Dr. Lavinia Alberi (SICHH, Fribourg, Switzerland). For all subjects in this study, amyloid imaging was used to differentiate between control subjects and AD cases, with control subjects lacking any amyloid deposition and AD cases positive for amyloid deposition.

Human Blood Plasma samples were obtained from Australian Imaging, Biomarker & Lifestyle Flagship Study of Ageing (AIBL). The samples consist of control, MCI, AD, classified using a variety of cognitive assessments (e.g. MMSE) and PET imaging to determine amyloid status.

### Protein Determination

Protein concentrations in the samples were measured using the Pierce™ 660 nm Protein Assay (Thermo Scientific, 22660). Briefly, a serial dilution (0 to 2 mg/ml) was made from a 10 mg/ml stock of bovine serum albumin (BSA, Sigma, A9418). Three replicates of each BSA concentration were prepared by transferring 10 μl of the protein into a clear 96 well plate (Starlabs, E2996-1600). Samples were diluted with three concentrations (1:1, 1:2, 1:10) and three replicates of each concentration were placed into the same 96 well plate with each replicate containing 10 μl of sample. Subsequently, 150 μl of Pearce Reagent was added to the standards and samples and the mixture was left to incubate for 5 min with gentle shaking at room temperature (RT). Finally, the plate was read on a spectrophotometer (Molecular Devices, Versa Max) at 660 nm. The protein concentrations of the samples were determined using the slope and y-intercept from the BSA standard curve, both calculated with Microsoft Excel.

### ELISA

The standard curves and samples were run in triplicate. The human brain homogenate samples were diluted 1:160.The standard curve for determination of T14 peptide in tissue samples was diluted in PBS buffer and ranged from 8 to 100 nM of T14. Briefly, NUNC 96-well immunoplates (Sigma, M9410) were coated with 100 μl/well of sample or standard T14, covered with parafilm and incubated overnight at 4° C. with gentle shaking. The following day, the sample was removed by flicking the plate over a sink with running water, and 200 μl of the blocking solution containing 2% BSA in Tris-buffered saline and Tween 20 (TBS-T) were added and incubated for 4 h at RT with gentle shaking. Blocking solution was then removed and 100 μl of antibody, diluted in blocking solution to 1 μg/ml, was added and incubated overnight at 4° C. with gentle shaking. The primary antibody was removed the next day and wells were washed 3 times with 200 μl of TBS-T. After 100 μl of secondary enzyme-conjugated antibody diluted in blocking solution to 0.1 μg/ml were added and incubated for 2 h at RT with gentle shaking; the plate was covered with parafilm during all incubations. After 2 h, the plate was washed 4 times with TBS-T. The addition of 3,3,5,5-tetramethylbenzidine (Thermo Scientific, 34028) started the colour reaction. The reaction was stopped 30 min later with stopping solution containing 2 M H₂SO₄, and the absorbance was measured at 450 nm in a Vmax plate reader (Molecular Devices, Wokingham, UK).

### AlphaLISA Detection of T14-Alpha-7 Complex

Samples were extracted from homogenized human brain tissue using PerkinElmer lysis buffer and the protein concentration determined using the BCA method. For 100 mg tissue homogenization, 1 mL of lysis buffer was used. Five cycles of 40 second pulse, and 10 second breaks, on a shielded homogenizer were used for each sample. Samples were centrifuged at 40° C., 15000 rpm (15 minutes) for supernatants; these were diluted in PerkinElmer Assay buffer and used to measure T14-alpha-7 nicotinic receptor complexes in the presence of NBP14 (concentrations 0.065 μM-900 μM) with AlphaLISA following the manufacturer's protocol. The antibodies were biotinylated BTX on SA-donor beads and anti-rabbit T14 on acceptor beads; results read in an AlphaLISA Reader.

### Western Blotting

### Polyacrylamide Gel Electrophoresis of Protein Samples

Polyacrylamide gels (mini-PROTEAN® TGX stain free™ gels, 4-20%, 4568093 and 4561094, BIO-RAD, Watford, UK) were placed into the electrophoresis tank (mini-PROTEAN tetra system, BIO-RAD) and running buffer (25 mM TRIS-base, pH 8.6, 192 mM glycine, 0.1% SDS) was added to the gel and tank reservoirs. 30 μg of protein samples were prepared by mixing with distilled water and 4× Laemmli Sample Buffer (161-0747, BIO-RAD) and 2.5% mercaptoethanol (161-0710, BIO-RAD). Sample equivalent concentration of synthetic T14 was also prepared, which acted as the positive control for measuring endogenous T14 peptide. The samples were heated at 95° C. for 5 min before cooling on ice. 25 μg of samples and the positive control were loaded into the gels and were electrophoresed alongside a molecular weight marker (Precision Plus Protein™ Dual Xtra Standards, 161-0377, BIO-RAD) at 35 mA for 60 min. An ice block was placed inside the running tank to prevent any overheating.

### Transfer of Protein Samples Onto PVDF Membrane

Gels were transferred onto PVDF transfer membrane (88518, Fisher Scientific, Loughborough, UK) in a Mini Transblot Cell (BIO-RAD). Briefly, the PVDF transfer membrane was activated by soaking with methanol for 1 min followed by soaking with distilled water for 2 min. All layers were subsequently saturated with transfer buffer (20 mM TRIS-base pH 8.6, 154 mM glycine, 0.8% w/v SDS and 20% methanol). The transfer sandwich was placed into a transfer cassette, which was inserted into the mini transblot cell filled with transfer buffer. Finally, electrophoretic transfer took place for 90 min at 200 mA. An ice block was placed inside the transfer tank to prevent any overheating.

### Staining of PVDF Membrane

BLOT-Faststain™ (786-34, G-Biosciences, St. Louis, USA) was used to stain for total protein, acting as the loading control (Collins et al., 2015). Immediately after electrophoretic transfer, the PVDF transfer membrane was stained with the diluted BLOT-Faststain™ fixer solution (10-fold) for 2 min with gentle shaking. The membrane was then incubated with the diluted BLOT-Faststain™ developer solution (4-fold) for 1 min with gentle shaking. Subsequently, the membrane was stored at 4° C. in the dark in the developer solution for 30 min to allow protein bands to reach maximum intensity. Finally, the membrane was washed with cold distilled water to eliminate background staining and imaged using the G box (Syngene, Cambridge, UK). The membrane can then be destained using warm deionized water (40-45° C.) and made ready for the blocking stage.

### Detection of Protein Bands

The PVDF transfer membrane was blocked in TBS (TRIS-buffered saline, 20 mM

TRIS-base pH 7.5, 0.5 mM NaCl) containing 5% skimmed milk powder for 1 h, then washed twice for 7 min each in TTBS (TBS supplemented with 0.05% v/v Tween-20, P9416, Sigma-Aldrich, Gillingham, UK). The membrane was incubated overnight at 4° C. with anti-T14 antibody (rabbit polyclonal, made by Genosphere, Ashford, UK) diluted 1:1000 in TTBS containing 1% skimmed milk powder. On the following day, the primary antibody was removed. The membrane was washed three times for 5 min in TTBS, then incubated for 1 h at room temperature with the secondary antibody. The secondary antibody was goat anti rabbit IgG conjugated to HRP (ab6721, 1:5000, Abcam, Cambridge, UK), diluted in TTBS containing 1% milk. After secondary antibody incubation, membranes were washed three times for 5 min with TTBS before a final 10 min wash in TBS. T14 bands were detected using the G box (Syngene).

### Protein Band Imaging and Data Analysis

The PVDF membrane was placed in the G box (Syngene). Luminol and Peroxide solutions from Clarity™ Western ECL substrate (1705061, BIO-RAD) were mixed in equal parts and applied to the membrane. Images were taken in the dark at 1 min time intervals for 5 min to obtain the optimal signal for the T14 bands. Following that, the membrane was exposed to white light using an automatic setting in order to obtain an image for the molecular ladder. The blot images were then analysed using Image J. Boxes of equal size were placed around individual T14 bands in each lane, allowing measurement of individual band intensities. T14 all bands were analysed separately by placing boxes of equal sizes around each whole lane, thus measuring the intensities of all T14 bands. The resulting T14 individual and T14 all band intensities were subsequently divided by the total protein signals (loading controls), and then expressed as percentages of control subjects. Further analysis was carried out in Graphpad software (Graphpad prism 6, San Diego, CA).

### Statistical Analysis

All data analyzed in this paper were processed and plotted using Graphpad Instat (Graphpad prism 6). For comparison between two groups, unpaired two tailed t-tests were conducted. For correlation analysis between two variables, linear correlation was fitted with the r² and p values shown. All statistical significance was taken at a p value <0.05.

### Example 1—T14 Levels and Braak Staging

T14 is released within the brain and eventually into CSF, where it aggregates at any of six weights ranging from 25 KDa to 130 KDa. Despite the relatively small sample size (n=19), three of these significantly enhanced aggregated forms of T14 in post-mortem CSF were significantly correlated with the respective Braak staging. Two of these bands (25 KDa and 40 KDa), shown in FIG. 1, were also significantly increased in CSF from both living patients.

The profiling of T14 in CSF of living and post mortem Alzheimer's cases is similar, and if the latter correlates significantly with Braak staging, then the T14 profile enables the extrapolation of the Braak status in lumbar punctures from living patients. Therefore, the inventors have demonstrated that, in the long-term, T14 profiling can offer the prospect of an Alzheimer's disease biomarker for reading-out, during life, the ongoing status of individual neurodegeneration. Furthermore, the inventors believe that it will also be possible to link the CSF T14 profile with that in plasma, such that a routine blood test could be used to ascertain the ongoing status of the neurodegeneration in the brain, as it is happening.

### Example 2—Plasma T14 Levels and Amyloid PET Scanning

This study was based exclusively on the two sets of samples provided by AIBL (I+2), since only they were characterised by amyloid PET scans. An ELISA assay was used which measures the soluble T14 in its native, 3-dimensional form and has the eventual advantage that it is rapid, and readily quantifiable. The disadvantage is currently that some signal can be lost, and in varying amounts, across samples, due to absorption. However, a comparison revealed a significant correlation between plasma T14 levels and amyloid PET scan status, surprisingly showing that ELISA can discriminate positive vs negative brain amyloid (see FIG. 2).

### Example 3—Plasma T14 Levels and Behavioural Tests of Pathology

Western Blot assay measures denatured (i.e. boiled) T14 so that it can be run on gels. In contrast to ELISA, which measures soluble T14, Western Blots provide a read-out of aggregated T14, i.e. peptide bound either to itself, and/or to various other proteins of varying molecular weights present in plasma or CSF. This procedure is slower than ELISA and is less readily quantifiable, but has the advantage of relatively greater sensitivity, since the signal will not be so impacted by absorption. The results surprisingly show that Western blot of the same plasma samples as in example 2 can discriminate pathological brains from normal (FIG. 3).

By combining example 2 and 3, it is possible to allocate subjects into 1 of 3 non-overlapping groups, as shown in FIG. 4. The ultimate goal is to develop a prognostic test. A subsequent study explored the possibility that T14 levels can be used as a predictor of future cognitive impairment by comparing the values of blood sampled at the same time from within a cognitively normal cohort, but where several years later, a few had subsequently developed MCI or AD.

The T14 levels of those who had progressed later to show mental deterioration, were already showing a trend that deviated from the normal majority, as shown in FIG. 5.

### Example 4—Plasma Levels and MMSE Scores

The data shown in FIG. 7 show the levels of T14 detected by Western Blot in a single sample correlated to the MMSE at the time when the blood was taken (3 groups: >26, 20-25 and <20). When the cognitive impairment was sufficiently advanced to reveal a low (<20) MMSE score, the levels of T14 correlated significantly. A similar conclusion is reached when T14 levels detected by Western Blot in single samples are correlated with the degree of cognitive impairment based on basal MMSE score (3 groups: >26, 20-25 and <20) (see FIG. 8).

Using two different types of analyses, there is a surprisingly significant correlation between T14, measured in single samples, and MMSE, only when the starting score was already far advanced and the patient thus showed significant cognitive impairment. This finding does not reflect the sensitivity of the T14 test, but rather indicates the insensitivity of MMSE in the early stages of AD, as shown in the sigmoid curve in FIGS. 7 and 8.

### Example 5—Plasma T14 Levels and MMSE Scores Over Time

This study was based on samples exclusively from the Austrian clinic NeuroScios. A blood sample was taken at the time of diagnosis and subsequently 6 months, 12 months and 24 months thereafter. MMSE values were supplied and levels of T14 all bands were measured for each time point.

TABLE 1

MMSE change vs T14 change

| | R² | p | n | slope |
|---|---|---|---|---|
| 6 month | 0.01873 | 0.1619 | 106 | 0.009860 |
| 12 month | 0.004692 | 0.5027 | 98 | 0.005101 |
| 24 month | 0.05705 | 0.0553 | 65 | −0.01295 |

Table 1 shows the degree of MMSE change (=MMSE at initial AD diagnosis−MMSE at each time point after the diagnosis) correlated with levels of T14 change (=levels of T14 all bands at initial AD diagnosis−levels of T14 all bands at each time point after the diagnosis) for each time point after diagnosis for all patients samples provided by the clinic. When the condition is sufficiently advanced to show the greatest change in MMSE, is there a correlation with T14 concentration for each individual patient.

Once again, these results confirm that T14 plasma levels surprisingly correspond closely to MMSE scores, when AD is sufficiently advanced, i.e. when MMSE scores are sufficiently sensitive.

### Example 6—T14 in the Post-Mortem Alzheimer's Disease Brain

As illustrated in FIG. 9A, the inventors have demonstrated that there is a significant increase in the concentration of hippocampal T14 (normalised to Braak stage 0-II), at Braak stage VI. The inventors also investigated the binding of T14 to the alpha-7 receptor with AlphaLISA. As illustrated in FIG. 9B, this revealed a greater level of T14-binding in late-stage Alzheimer's disease (Braak V-VI) compared with Braak stages I-II.

Additionally, as illustrated in FIG. 9C, Western Blots of the Alzheimer's disease hippocampus showed a single T14-reactive band that increased approximately 2-fold from early (Braak 0-II) to late stages (Braak V-VI). Surprisingly, the data also shows that changes in T14 levels can be used to determine the early Braak stages, I and II. Importantly, therefore, this demonstrates that the concentration of T14 can be used to determine the Braak stage for living asymptomatic patients (i.e. those in Braak stage I and II).

### Conclusions

Example 1 clearly shows that the T14 profile enables extrapolation of the Braak status in lumbar punctures from living patients. This is the first report that Braak staging can be conducted on a living patient, rather than the current method performed on post-mortem examination of the brain during autopsy. Accordingly, T14 profiling can offer the prospect of an AD biomarker for reading-out, during life, the ongoing status of individual neurodegeneration and/or cognitive decline. It is also possible to link CSF T14 profiles with that in plasma, and a routine blood test can be used to ascertain the ongoing status of the neurodegeneration in the brain, as it is happening.

Example 2 provides further evidence that the T14 test can be used to identify those who are amyloid positive, without the need for a PET scan. This means, therefore, that testing for T14 enables a clinician to decide whether or not a PET scan is required, and avoids the current problem that certain patients are unnecessarily subjected to a PET scan.

Examples 3, 4 and 5 show that it is surprisingly possible to distinguish between patients who are cognitively normal and those with cognitive impairment. Taken together, these two tests on a single blood sample will provide a diagnostic of potential value to identify cohorts for drug screening and eventual treatment, i.e. as a diagnostic marker.

Additionally, Example 6 demonstrates that it is possible to determine the earlier Braak stages I and II, by detecting changes in T14 levels. Advantageously, this allows the identification of earlier Braak stages (i.e. I or II) in asymptomatic patients, who can therefore be treated during the early stages of Alzheimer's disease in order to prevent or slow its progression.

In summary, the T14 test shows sensitivity and a link to pathology in three of the main benchmarks for AD (amyloid imaging, MMSE/behavioural scores and Braak staging). To date, none of these parameters have proved satisfactory for predicting cognitive impairment. Accordingly, T14 represents a first-in-class prognostic plasma biomarker. The above results suggest that measurement of T14 in plasma should and could now be used as a diagnostic and prognostic marker for Alzheimer's disease.

1. A method of determining the Braak stage of a living subject, the method comprising:
(a) analysing, in a sample obtained from a living test subject, the concentration of (i) a soluble peptide comprising or consisting of SEQ ID No:3 (T14), or a variant or fragment thereof and/or (ii) an aggregated peptide comprising or consisting of SEQ ID No:3, or a variant or fragment thereof; and
(b) comparing this concentration with a reference value from a control population of deceased subjects having known Braak stages for concentrations of either soluble or aggregated peptide comprising or consisting of SEQ ID No:3, or a variant or fragment thereof,
wherein the Braak stage of the living test subject is determined by comparing the concentration of either the soluble or aggregated peptide comprising or consisting of SEQ ID No:3, or a variant or fragment thereof, with the respective reference value that is associated with a Braak stage.

2. A method according to claim 1, wherein a lower concentration of soluble peptide comprising or consisting of SEQ ID No:3, or a variant or fragment thereof, compared to the reference value, is indicative of a later Braak stage, optionally wherein the sample is blood plasma.

3. A method according to any preceding claim, wherein a higher concentration of aggregated peptide comprising or consisting of SEQ ID No:3, or a variant or fragment thereof, compared to the reference value, is indicative of a later Braak stage, optionally wherein the sample is CSF.

4. A Braak staging kit, for determining the Braak stage of a living subject, the kit comprising:
(a) means for determining, in a sample obtained from a test subject, the concentration of (i) a soluble peptide comprising or consisting of SEQ ID No:3, or a variant or fragment and/or (ii) an aggregated peptide comprising or consisting of SEQ ID No:3, or a variant or fragment thereof; and
(b) a reference value from a control population of deceased subjects having known Braak stages for concentrations of either soluble or aggregated peptide comprising or consisting of SEQ ID No:3, or a variant or fragment thereof,
wherein the kit is used to identify the Braak stage of the living subject by comparing the concentration of either soluble or aggregated peptide comprising or consisting of SEQ ID No:3, or a variant or fragment thereof, with the respective reference value that is associated with a Braak stage.

5. Use of a peptide comprising or consisting of SEQ ID No:3, or a variant or fragment thereof, as a biomarker for determining the Braak stage of a living subject.

6. A method of determining if a subject should receive a positron emission tomography (PET) scan, the method comprising:
(a) analysing, in a sample obtained from a test subject, the concentration of (i) a soluble peptide comprising or consisting of SEQ ID No:3 (T14), or a variant or fragment thereof and/or (ii) an aggregated peptide comprising or consisting of SEQ ID No:3, or a variant or fragment thereof; and
(b) comparing this concentration with a reference value from a control population for concentrations of either a soluble or aggregated peptide comprising or consisting of SEQ ID No:3, or a variant or fragment thereof,
wherein a lower concentration of soluble peptide comprising or consisting of SEQ ID No:3, or a variant or fragment thereof, or an altered concentration of aggregated peptide comprising or consisting of SEQ ID No:3, or a variant or fragment thereof, compared to the respective reference value is indicative that the subject should receive a PET scan.

7. A method according to claim 6, wherein a lower concentration of a soluble peptide comprising or consisting of SEQ ID No:3 or a variant or fragment thereof compared to the reference value is indicative that the patient is beta amyloid positive, and/or a higher concentration of a soluble peptide comprising or consisting of SEQ ID No:3 or a variant or fragment thereof compared to the reference value is indicative that the patient is beta amyloid negative, optionally wherein the sample is blood plasma.

8. A method according to either claim 6 or claim 7, wherein a lower concentration of a soluble peptide comprising or consisting of SEQ ID No:3 or a variant or fragment thereof compared to the reference value is indicative that the subject is cognitively impaired, and/or a higher concentration of a soluble peptide comprising or consisting of SEQ ID No:3 or a variant or fragment thereof compared to the reference value is indicative that the patient is cognitively normal, optionally wherein the sample is blood plasma.

9. A method according to any one of claims 6-8, wherein when the sample is CSF, a higher concentration of an aggregated peptide comprising or consisting of SEQ ID No:3 or a variant or fragment thereof compared to the reference value is indicative that the patient is beta amyloid positive, and/or a lower concentration of an aggregated peptide comprising or consisting of SEQ ID No:3 or a variant or fragment thereof compared to the reference value is indicative that the patient is beta amyloid negative.

10. A method according to any one of claims 6-9, wherein when the sample is blood plasma, a lower concentration of an aggregated peptide comprising or consisting of SEQ ID No:3 or a variant or fragment thereof compared to the reference value is indicative that the patient is beta amyloid positive, and/or a higher concentration of an aggregated peptide comprising or consisting of SEQ ID No:3 or a variant or fragment thereof compared to the reference value is indicative that the patient is beta amyloid negative.

11. A method according to any one of claims 6-10, wherein a lower concentration of an aggregated peptide comprising or consisting of SEQ ID No:3 or a variant or fragment thereof is indicative that the subject is cognitively impaired, and/or a higher concentration of an aggregated peptide comprising or consisting of SEQ ID No:3 or a variant or fragment thereof is indicative that the patient is cognitively normal.

12. A PET scan determining kit, for determining if a subject should receive a PET scan, the kit comprising:
(a) means for determining, in a sample obtained from a test subject, the concentration of (i) a soluble peptide comprising or consisting of SEQ ID No:3, or a variant or fragment and/or (ii) an aggregated peptide comprising or consisting of SEQ ID No:3, or a variant or fragment thereof; and (b) a reference value from a control population for concentrations of soluble or aggregated peptide comprising or consisting of SEQ ID No:3, or a variant or fragment thereof,
wherein the kit is used to identify a lower concentration of soluble peptide comprising or consisting of SEQ ID No:3, and/or an altered concentration of aggregated peptide comprising or consisting of SEQ ID No:3, in the sample from the test subject, compared to the respective reference value, thereby suggesting that the subject should receive a PET scan.

13. Use of a peptide comprising or consisting of SEQ ID No:3, or a variant or fragment thereof, as a biomarker for determining if a subject requires a PET scan.

14. A method, kit or use according to any preceding claim, wherein the subject has, or is suspected of having, a neurodegenerative disease selected from a group consisting of: Alzheimer's disease; Parkinson's disease; Huntington's disease; Motor Neurone disease; Spinocerebellar type 1, type 2, and type 3; Amyotrophic Lateral Sclerosis (ALS); schizophrenia; Lewy-body dementia; and Frontotemporal Dementia. It is preferred, however, that the invention is used to study or predict cognitive decline in any neurological disorder associated with non-enzymatic function of AChE, in particular, for example, Alzheimer's Disease, Parkinson's Disease and Motor Neuron Disease, and preferably Alzheimer's Disease and Parkinson's Disease.

15. A method, kit or use according to any preceding claim, wherein the subject has, or is suspected of having, Alzheimer's Disease.

16. A method, kit or use according to any preceding claim, comprising detection of soluble and/or aggregated peptide comprising or consisting of SEQ ID No:3.

17. A method, kit or use according to any preceding claim, comprising detection of soluble and/or aggregated peptide comprising or consisting of one or more of any of T7-T13 (i.e. SEQ ID No: 4-10).

18. A method, kit or use according to any preceding claim, wherein the sample is blood, plasma, serum, spinal fluid, urine, sweat, saliva, tears, breast aspirate, prostate fluid, seminal fluid, vaginal fluid, stool, cervical scraping, cytes, amniotic fluid, intraocular fluid, mucous, moisture in breath, animal tissue, cell lysates, tumour tissue, hair, skin, buccal scrapings, lymph, interstitial fluid, nails, bone marrow, cartilage, prions, bone powder, ear wax, or combinations thereof.

19. A method, kit or use according to any preceding claim, wherein the sample comprises blood, urine, tissue, or CSF etc.

20. A method, kit or use according to any preceding claim, wherein the sample comprises a blood sample, optionally blood plasma.

21. A method, kit or use according to any preceding claim, wherein an immunoassay is employed to measure T14 (SEQ ID No:3) peptide levels.

22. A method, kit or use according to any preceding claim, wherein soluble peptide SEQ ID No:3 (T14), or a variant or fragment thereof, is determined using ELISA.

23. A method, kit or use according to any preceding claim, wherein aggregated peptide SEQ ID No:3 (T14), or a variant or fragment thereof, is determined using Western Blot.

24. A method, kit or use according to any preceding claim, wherein means for determining, in the sample obtained from the test subject, the concentration of (i) a soluble T14 and/or (ii) an aggregated T14 comprises an anti-T14 antibody or antigen-binding fragment thereof.

25. A method, kit or use according to claim 24, wherein the antibody or antigen-binding fragment thereof specifically binds to SEQ ID No:3, optionally one or more amino acid in SEQ ID No: 11, optionally wherein the antibody or antigen-binding fragment thereof does not bind to SEQ ID No:2 (i.e. T30), SEQ ID No:13 (i.e. T15) and/or SEQ ID No: 14.

26. A method, kit or use according to any preceding claim, wherein:
(i) a lower concentration of soluble peptide comprising or consisting of SEQ ID No:3, or a variant or fragment thereof, compared to the reference value is indicative of Braak stage I;
(ii) a lower concentration of soluble peptide comprising or consisting of SEQ ID No:3, or a variant or fragment thereof, compared to the reference value is indicative of Braak stage II; and/or
(iii) a lower concentration of soluble peptide comprising or consisting of SEQ ID No:3, or a variant or fragment thereof, compared to the reference value is indicative of Braak stage III.

27. A method, kit or use according to any preceding claim, wherein when the sample is CSF:
(i) a higher concentration of aggregated peptide comprising or consisting of SEQ ID No:3, or a variant or fragment thereof, compared to the reference value is indicative of Braak stage I;
(ii) a higher concentration of aggregated peptide comprising or consisting of SEQ ID No:3, or a variant or fragment thereof, compared to the reference value is indicative of Braak stage II; and/or
(iii) a higher concentration of aggregated peptide comprising or consisting of SEQ ID No:3, or a variant or fragment thereof, compared to the reference value is indicative of Braak stage III.

28. A method, kit or use according to any preceding claim, wherein when the sample is blood plasma:
(i) a lower concentration of aggregated peptide comprising or consisting of SEQ ID No:3, or a variant or fragment thereof, compared to the reference value is indicative of Braak stage I;
(ii) a lower concentration of aggregated peptide comprising or consisting of SEQ ID No:3, or a variant or fragment thereof, compared to the reference value is indicative of Braak stage II; and/or
(iii) a lower concentration of aggregated peptide comprising or consisting of SEQ ID No:3, or a variant or fragment thereof, compared to the reference value is indicative of Braak stage III.


SEQUENCE LISTING (14 sequences)

SEQ ID NO: 1 - protein, 614 aa, Homo sapiens
MRPPQCLLHTPSLASPLLLLLLWLLGGGVGAEGREDAELLVTVRGGRLRGIRLKTPGGPVSAFLGIPFAEPPMGPRRFLPPEPKQPWSGVVDATTFQSVCYQYVDTLYPGFEGTEMWNPNRELSEDCLYLNVWTPYPRPTSPTPVLVWIYGGGFYSGASSLDVYDGRFLVQAERTVLVSMNYRVGAFGFLALPGSREAPGNVGLLDQRLALQWVQENVAAFGGDPTSVTLFGESAGAASVGMHLLSPPSRGLFHRAVLQSGAPNGPWATVGMGEARRRATQLAHLVGCPPGGTGGNDTELVACLRTRPAQVLVNHEWHVLPQESVFRFSFVPVVDGDFLSDTPEALINAGDFHGLQVLVGVVKDEGSYFLVYGAPGFSKDNESLISRAEFLAGVRVGVPQVSDLAAEAVVLHYTDWLHPEDPARLREALSDVVGDHNVVCPVAQLAGRLAAQGARVYAYVFEHRASTLSWPLWMGVPHGYEIEFIFGIPLDPSRNYTAEEKIFAQRLMRYWANFARTGDPNEPRDPKAPQWPPYTAGAQQYVSLDLRPLEVRRGLRAQACAFWNRFLPKLLSATDTLDEAERQWKAEFHRWSSYMVHWKNQFDHYSKQDRCSDL

SEQ ID NO: 2 - protein, 30 aa, Homo sapiens
KAEFHRWSSYMVHWKNQFDHYSKQDRCSDL

SEQ ID NO: 3 - protein, 14 aa, Homo sapiens
AEFHRWSSYMVHWK

SEQ ID NO: 4 - protein, 7 aa, Homo sapiens
SYMVHWK

SEQ ID NO: 5 - protein, 8 aa, Homo sapiens
SSYMVHWK

SEQ ID NO: 6 - protein, 9 aa, Homo sapiens
WSSYMVHWK

SEQ ID NO: 7 - protein, 10 aa, Homo sapiens
RWSSYMVHWK

SEQ ID NO: 8 - protein, 11 aa, Homo sapiens
HRWSSYMVHWK

SEQ ID NO: 9 - protein, 12 aa, Homo sapiens
FHRWSSYMVHWK

SEQ ID NO: 10 - protein, 13 aa, Homo sapiens
EFHRWSSYMVHWK

SEQ ID NO: 11 - protein, 7 aa, Homo sapiens
SYMVHWK

SEQ ID NO: 12 - protein, 4 aa, Homo sapiens
VHWK

SEQ ID NO: 13 - protein, 15 aa, Homo sapiens
NQFDHYSKQDRCSDL

SEQ ID NO: 14 - protein, 42 aa, Homo sapiens [sequence omitted]